(19) United States
(12) Patent Application Publication
(10) Pub. No.: US 2020/0237552 A1
(43) Pub. Date: Jul. 30, 2020

(54) MALE HYGIENE PRODUCT AND PACKAGE THEREOF

(71) Applicant: CLW Consulting, Woodbury, MN (US)

(72) Inventor: Charles WHITE, Woodbury, MN (US)

(21) Appl. No.: 16/727,918

(22) Filed: Dec. 27, 2019

Related U.S. Application Data
(60) Provisional application No. 62/796,662, filed on Jan. 25, 2019.

(51) Int. Cl. A61F 6/00; A61F 6/04; A45C 11/24

(52) U.S. Cl. CPC A61F 6/005; A45C 2011/007; A45C 11/24; A61F 6/04

(57) ABSTRACT

Disclosed is a male hygiene product. The male hygiene product includes a first packet containing a cleaning article therein and a second packet containing a condom therein. The first packet and the second packet are packaged in a manner such that the cleaning article and the condom are accessible together.

### TECHNICAL FIELD

The present disclosure relates generally to hygiene products and, more particularly to, a male hygiene product and a package thereof.

### BACKGROUND

The condoms are primarily used as a contraceptive or as a protection against infection. However, it has been observed that after sexual intercourse cleaning of semen or body parts is performed with the help of items that are generally available or handy. Typically, most of the items available and used for cleaning are either non-hygienic, unsensitized, lacks absorbency, flexibility and odor-eliminating properties. This may cause health issues or cleaning problems, such as stained cloths or bed, i.e. causing unhealthy and unpleasant sexual intercourse experiences. Additionally, this may cause psychological stress during the intercourse affecting an overall sexual performance and the sexual intercourse experience.

In light of the foregoing discussion, there exists a need to overcome the aforementioned drawbacks associated with unhealthy and unpleasant sexual intercourse experiences.

### SUMMARY

Various embodiments of the present disclosure provide a male hygiene product.

In an embodiment, a male hygiene product is disclosed. The male hygiene product includes a first packet containing a cleaning article therein and a second packet containing a condom therein. The first packet and the second packet are packaged in a manner such that the cleaning article and the condom are accessible together.

In another embodiment, a male hygiene product is disclosed. The male hygiene product includes a first packet containing a cleaning article therein and a second packet containing a condom therein. The first packet and the second packet are packaged in a manner such that the cleaning article and the condom are accessible together. The male hygiene product also includes a third packet containing the first and second packets therein.

In yet another embodiment, a package of male hygiene product is disclosed. The package includes a plurality of male hygiene products. Each product includes a first packet containing a cleaning article therein, and a second packet containing a condom therein. The first packet and the second packet are packaged in a manner such that the cleaning article and the condom are accessible together. The package also includes a package cover containing the plurality of male hygiene products therein.

Other aspects and example embodiments are provided in the drawings and the detailed description that follows.

### BRIEF DESCRIPTION OF THE FIGURES

For a more complete understanding of example embodiments of the present technology, reference is now made to the following descriptions taken in connection with the accompanying drawings in which:

FIG. 1 is a schematic drawing of a male hygiene product, in accordance with an embodiment of the present disclosure;

FIG. 2 is a schematic drawing of the male hygiene product of FIG. 1 in a folded state, in accordance with an embodiment of the present disclosure; and

FIG. 3 is a schematic drawing of a male hygiene product, in accordance with another embodiment of the present disclosure; and

FIG. 4 is a perspective schematic drawing of a package of a male hygiene product, in accordance with another embodiment of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details.

Reference in this specification to “one embodiment” or “an embodiment” means that a particular feature, structure, or characteristic described in connection with the embodiment is included in at least one embodiment of the present disclosure. The appearance of the phrase “in an embodiment” in various places in the specification is not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

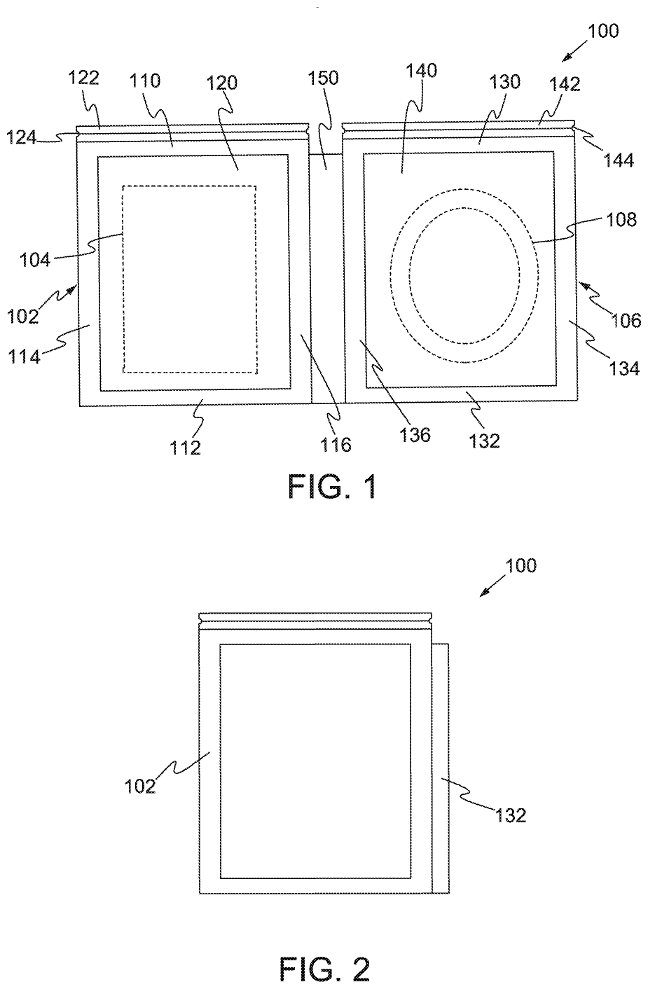

Referring now to the drawings, FIG. 1 is a schematic drawing of a male hygiene product 100, in accordance with an embodiment of the present disclosure. As shown, the male hygiene product 100 includes a first packet 102 containing a cleaning article 104 therein, and a second packet 106 containing a condom 108 therein.

The first packet 102 includes a top wall 110, a bottom wall 112, a first side wall 114 and a second side wall 116. The bottom wall 112, the first side wall 114 and the second side wall 116 are sealed along it's inner edges to form a first cavity 120 for containing the cleaning article 104 therein. In other words, the first packet 102 is a hollow pouch made of a pair of spaced apart layers, that are sealed along three sides to form three walls, such as the bottom wall 112, the first side wall 114 and the second side wall 116, and a cavity, such as the first cavity 120, surrounded by the three walls. The hollow pouch (interchangeably referred to as the first packet 102) also includes a fourth side, such as the top wall 110, formed by the unsealed pair of spaced apart layers to provide access to the cavity. As shown and explained, it will be evident that the first packet 102 has a rectangular shape, therefor includes four walls. However, alternatively, the first packet 102 may be configured to have other shapes such as a circular, an oval or any other polygonal shape, such as square, hexagonal and so forth.

The first packet 102 also includes a tear strip 122 having a tear guiding region 124. As shown, the tear strip 122 is configured over the top wall 110, i.e. the tear strip 122 runs along an entire length of the top wall 110. The tear strip 122 openably seals the top wall 110, and thereby provides access to the cleaning article 104 contained in the first cavity 120. In use, a tear strip 122 can be held with two hands and pressure can be exerted on the tear guiding region 124 by moving the hands away from each other and thereby tearing the tear strip 122 along the tear guiding region 124. This will cause the top wall 110 to open for providing access to the cleaning article 104.

According to an embodiment, the cleaning article 104 is one of a wipe, a piece of cloth or a piece of paper. In particular, the cleaning article 104 is one of a wipe or a disposable cloth or paper treated with a cleansing agent, medically compatible for cleaning body part. In simple words, the cleaning article 104 may be a plain piece of cloth or paper infused with or without any cleansing agent.

As mentioned herein above, the second packet 106 (similar to the first packet 102) includes a top wall 130, a bottom wall 132, a first side wall 134 and a second side wall 136. The bottom wall 132, the first side wall 134 and the second side wall 136 are sealed along it's inner edges to form a second cavity 140 for containing the condom 108 therein. In other words, the second packet 106 is a hollow pouch made of a pair of spaced apart layers, that are sealed along three sides in a manner to form three walls, such as the bottom wall 132, the first side wall 134 and the second side wall 136, and a cavity, such as the second cavity 140, surrounded by the three walls. The hollow pouch (interchangeably referred to as the second packet 106) also includes a fourth side, such as the top wall 130, formed by the unsealed pair of spaced apart layers to provide access to the cavity. As shown and explained, it will be evident that the second packet 106 has a rectangular shape, therefor includes four walls. However, alternatively, the second packet 106 may be configured to have other shapes such as a circular, an oval or any other polygonal shape, such as square, hexagonal and so forth.

The second packet 106 also includes a tear strip 142 having a tear guiding region 144. As shown, the tear strip 142 is configured over the top wall 130, i.e. the tear strip 142 runs along an entire length of the top wall 130. The tear strip 142 openably seals the top wall 130, and thereby provides access to the condom 108 contained in the second cavity 140. In use, the tear strip 142 can be held with two hands and pressure can be exerted on the tear guiding region 144 by moving the hands away from each other and thereby tearing the tear strip 142 along the tear guiding region 144. This will cause the top wall 130 to open for providing access to the condom 108.

According to an embodiment, both the first and second packets 102, 106 are configured to have same shape and dimension. Alternatively, each of the first and second packets 102, 106 may be configured to have different shapes and dimensions.

In an embodiment, the first and second packets 102, 106 are foldably coupled to each other. More specifically, the first and second packets 102, 106 are coupled via a connecting strip 150 to fold there-along and overlap to form a double packet. In simple words, the first and second packets 102, 106 are foldably coupled via the connecting strip 150, i.e. the coupled first and second packets 102, 106 can overlap on each other (along the connecting strip 150, when folded) to form the double packet, i.e. the first and second packets 102, 106 lie on each other in a folded state. However, as shown in FIG. 1, the connecting strip 150 extends from the second side walls 116, 136 of the first and second packets 102, 106, such that the first and second packets 102, 106 are positioned side-by-side in a laid-out flat configuration.

According to an embodiment, the connecting strip 150 includes one of a hook-and-loop fasteners, bands, buttons, elastics, a zippers or a seam (not shown) for coupling the first and second packets 102, 106. Alternatively, the connecting strip 150 may not include any such fastening means (such as the hook-and-loop fasteners, bands, buttons, elastics, the zippers or the seam), rather merely connected by the connecting strip 150, which is made of a material from which the first and second packets 102, 106 are made. In another embodiment, the first and second packets 102, 106 may not be coupled by the connecting strip 150, rather the first and second packets 102, 106 merely glued to each other, for example, at their central portions or side portions, such as the first side walls 114, 134 or the second side walls 116, 136.

In an embodiment, the first and second packets 102, 106 (i.e. the hollow pouches) are made of a material selected from a group including plastic, rubber, paper, fabric and any combination thereof. Typically, the first and second packets 102, 106 are made of flexible sheets or layers of said material and configured to have a pouch like configuration. Alternatively, the first and second packets 102, 106 may be made of a non-flexible material, such as cardboard or corrugated sheets.

According to an embodiment, at least one of the first and second packets 102, 106 includes a printed information. The printed information may be an advertising content that is used for marketing purposes of the male hygiene product 100. Alternatively, the printed information may be product specification information, like details regarding the cleaning article 104 and the condom 108. Additionally, the printed information may be associated with instructions regarding how to use the male hygiene product 100.

The first and second packets 102, 106 are packaged in a manner such that the cleaning article 104 and the condom 108 are accessible together. The term “packaged” used herein relates to mechanical coupling or association between the first and second packet 102, 106 such that both the first and second packets 102, 106 remain accessible together. For example, in use, a user may tear-off the second packet 106 to take out the condom 108 for having sexual intercourse. Thereafter, the user may tear-off the first packet 102 to take out the cleaning article 104 for cleaning the male genitalia. Alternatively, the cleaning article 104 may be used for cleaning other male body part(s) or any body part of the partner's body.

Referring now to FIG. 2, illustrated is a schematic drawing of the male hygiene product 100 of FIG. 1 in a folded state, in accordance with an embodiment of the present disclosure. As shown, the first packet 102 overlaps onto the second packet 106 (not shown as being overlapped by the first packet 102) along the connecting strip 150 to form the double packet. Further, it will be appreciated that neither the cleaning article 104 nor the condom 108 is shown in FIG. 2 for purpose of clarity.

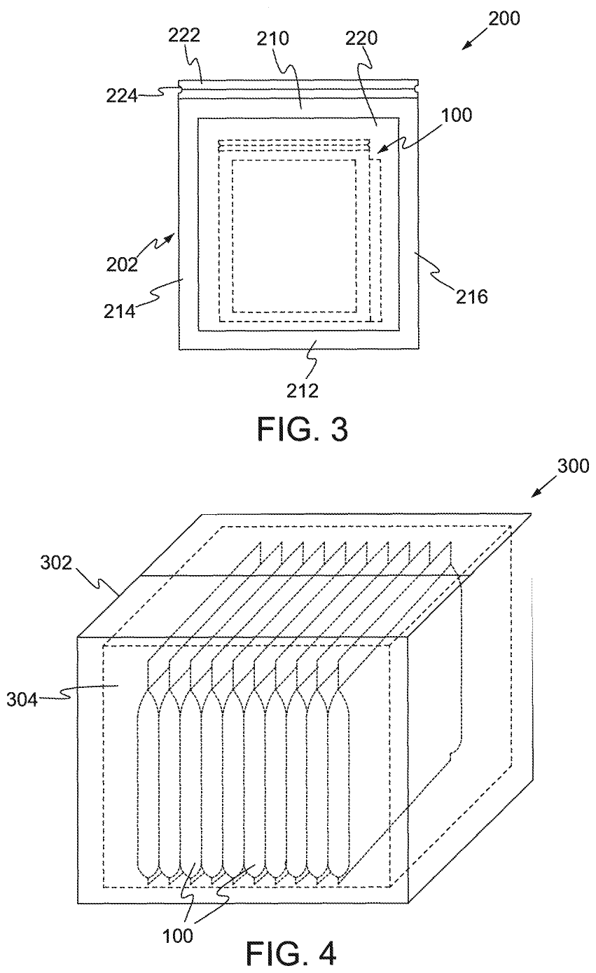

FIG. 3 is a schematic drawing of a male hygiene product 200, in accordance with another embodiment of the present disclosure. The male hygiene product 200 is substantially, structurally and functionally similar to the male hygiene product 100, explained in conjunction with FIG. 1. For example, the male hygiene product 200 also includes a first packet, such as the first packet 102 (shown in FIG. 1), containing a cleaning article, such as the cleaning article 104; and a second packet, such as the second packet 106, containing a condom, such as the condom 108. However, the male hygiene product 200 also includes a third packet 202 for containing the first and second packets therein in a folded state thereof. Specifically, it will be evident that, as shown in FIG. 3, the third packet 202 encloses the first and second packets in the folded state (i.e. the male hygiene product 100 as shown in FIG. 2).

The third packet 202 includes a top wall 210, a bottom wall 212, a first side wall 214 and a second side wall 216. The bottom wall 212, the first side wall 214 and the second side wall 216 are sealed along it's inner edges to form a third cavity 220 for containing the first and second packets (i.e. the male hygiene product 100 as shown in FIG. 2) therein in the folded state thereof. The third packet 202 also includes a tear strip 222, having a tear guiding region 224, configured over the top wall 210. The tear strip 222 openably seals the top wall 210, and thereby provides access to the first and second packets contained therein, when torn away.

Referring now to FIG. 4, illustrated is a perspective schematic drawing of a package 300 of a male hygiene product, in accordance with another embodiment of the present disclosure. The package 300 includes a plurality of male hygiene products, such as the male hygiene product 100, explained in conjunction with FIG. 1. The package 300 also includes a package cover 302 for containing the plurality of male hygiene products 100 therein. The package cover 302 includes a hollow space 304 for containing the plurality of male hygiene products 100 therein. Typically, the package cover 302 is used for packaging the plurality of male hygiene products 100. According to an embodiment, the package cover 302 may be made of paper, cardboard or corrugated sheets. Additionally, in the package 300 each of the plurality of male hygiene products 100 further includes a third packet, such as the third packet 202 (explained in conjunction with FIG. 3), for containing the first and second packets therein. Therefore, it will be apparent that the package 300 may include the plurality of male hygiene products, such as the male hygiene product 100 explained in conjunction with FIG. 1, or the male hygiene product 200 explained in conjunction with FIG. 3.

Embodiments of the present disclosure substantially eliminate or at least partially address the aforementioned problems in the background, and provide a male hygiene product. The male hygiene product includes a condom and a cleaning article packaged in a manner such that both the condom and the cleaning article are easily accessible together. This allows a user to have healthy and pleasant sexual intercourse experiences. Specifically, after using the condom the user can use the cleaning article for cleaning the male genitalia or other body part(s) or any body part of the partner's body. This allows the user and the partner to experience a clean, hygienic, odor-free and stress-free sexual intercourse experience. Additionally, the used condom and the cleaning article may be inserted back into the first and second packets of the male hygiene product for easy and suitable disposal thereof. Finally, due to the foldable nature of the male hygiene product, it is to carry and store.

The embodiments illustrated and described herein as well as embodiments not specifically described herein but within the scope of aspects of the invention constitute exemplary male hygiene products.

The benefits and advantages described above may relate to one embodiment or may relate to several embodiments. The embodiments are not limited to those that solve any or all of the stated problems or those that have any or all of the stated benefits and advantages.

Aspects of any of the examples described above may be combined with aspects of any of the other examples described to form further examples without losing the effect sought.

The above description is given by way of example only and various modifications may be made by those skilled in the art. The above specification, examples and data provide a complete description of the structure and use of exemplary embodiments. Although various embodiments have been described above with a certain degree of particularity, or with reference to one or more individual embodiments, those skilled in the art could make numerous alterations to the disclosed embodiments without departing from the spirit or scope of this specification.

What is claimed is:

1. A male hygiene product comprising:
a first packet containing a cleaning article therein; and
a second packet containing a condom therein;
wherein the first packet and the second packet are packaged in a manner such that the cleaning article and the condom are accessible together.

2. The male hygiene product as claimed in claim 1, wherein the first and second packets are foldably coupled to each other.

3. The male hygiene product as claimed in claim 2, wherein the first and second packets are coupled via a connecting strip to fold there-along and overlap to form a double packet.

4. The male hygiene product as claimed in claim 3, wherein the connecting strip comprises one of hook-and-loop fasteners, bands, buttons, elastics, a zippers or a seam for coupling the first and second packets.

5. The male hygiene product as claimed in claim 1, wherein the cleaning article is one of a wipe, a piece of cloth or a piece of paper.

6. The male hygiene product as claimed in claim 1, wherein each of the first and second packets is a hollow pouch.

7. The male hygiene product as claimed in claim 6, wherein the hollow pouch is made of a material selected from a group comprising plastic, rubber, paper, fabric and any combination thereof.

8. The male hygiene product as claimed in claim 6, wherein the hollow pouch is configured to have one of a circular, an oval or a polygonal shape.

9. The male hygiene product as claimed in claim 1, wherein each of the first and second packets comprises a tear strip having a tear guiding region.

10. The male hygiene product as claimed in claim 1, wherein at least one of the first and second packets comprises a printed information.

11. A male hygiene product comprising:
a first packet containing a cleaning article therein;
a second packet containing a condom therein, wherein the first packet and the second packet are packaged in a manner such that the cleaning article and the condom are accessible together; and
a third packet containing the first and second packets therein.

12. The male hygiene product as claimed in claim 11, wherein the first and second packets are foldably coupled via a connecting strip to fold there-along and overlap to form a double packet.

13. The male hygiene product as claimed in claim 12, wherein the connecting strip comprises one of hook-and-loop fasteners, bands, buttons, elastics, a zippers or a seam for coupling the first and second packets.

14. The male hygiene product as claimed in claim 12, wherein the cleaning article is one of a wipe, a piece of cloth or a piece of paper.

15. The male hygiene product as claimed in claim 12, wherein each of the first, second and third packets is a hollow pouch made of a material selected from a group comprising plastic, rubber, paper, fabric and any combination thereof.

16. The male hygiene product as claimed in claim 15, wherein the hollow pouch is configured to have one of a circular, an oval or a polygonal shape.

17. The male hygiene product as claimed in claim 12, wherein each of the first, second and third packets comprises a tear strip having a tear guiding region.

18. The male hygiene product as claimed in claim 11, wherein at least one of the first, second and third packets comprises a printed information.

19. A package of male hygiene product, the package comprising:
a plurality of male hygiene products, each comprising
a first packet containing a cleaning article therein;
a second packet containing a condom therein, wherein the first packet and the second packet are packaged in a manner such that the cleaning article and the condom are accessible together; and
a package cover containing the plurality of male hygiene products therein.

20. The package as claimed in claim 19, wherein each of the plurality of male hygiene products further comprises a third packet containing the first and second packets therein.
